(19) United States
(12) Patent Application Publication
(10) Pub. No.: US 2019/0276439 A1
(43) Pub. Date: Sep. 12, 2019

(54) METHOD OF TREATING SOLID TUMORS

(71) Applicant: Hutchison Medipharma Limited, Shanghai (CN)

(72) Inventors: Chuan QI, Shanghai (CN); Jianming XU, Shanghai (CN)

(21) Appl. No.: 16/462,194

(22) PCT Filed: Nov. 18, 2016
(86) PCT No.: PCT/CN2016/106404

§ 371 Date: May 17, 2019

(51) Int. Cl. C07D 403/12; A61K 31/513; A61P 35/00

(52) U.S. Cl. CPC C07D 403/12; A61P 35/00; A61K 31/513

(57) ABSTRACT

Disclosed are methods for treating a solid tumor, such as a neuroendocrine tumor, in a patient comprising administration to the patient in need thereof a therapeutically effective amount of a compound that is an inhibitor of VEGFR 1, 2, and 3, FGFR 1, and CSF1R, such as N-(2-(dimethylamino)ethyl)-1-(3-((4-((2-methyl-1H-indol-5-yl)oxy)pyrimidin-2-yl)amino)phenyl)-methanesulfonamide.

Vascular endothelial growth factor (VEGF)- and fibroblast growth factor (FGF)-mediated pathways play key roles in tumor angiogenesis (1, 2). Secretion of VEGF and FGF by tumor cells promotes rapid proliferation and packing of endothelial cells, which leads to the formation of excessive and coarsely packed blood vessels (3). These blood vessels supply oxygen and nutrients to the tumor and promote tumor cell leakage into the circulation, resulting in increased tumor growth and a risk of metastasis (3). While VEGF receptor (VEGFR)-targeted therapies are established as an important treatment option in the management of several cancer types, a significant number of patients do not respond to treatment or only respond for a relatively short time, due to the development of resistance mechanisms targeting alternative molecular pathways (4).

Evidence suggests that in response to treatment with anti-VEGF therapies, some tumors can increase secretion of FGF to stimulate endothelial cell proliferation, promote tumor angiogenesis, and bypass VEGF signaling pathways (4, 5). Furthermore, there is evidence of a role for VEGFR, FGF receptors (FGFRs), and colony stimulating factor 1 receptor (CSF1R, also known as Fms) in tumor immune evasion. VEGF secreted by tumors can activate VEGFR signaling pathways in T cells; this leads to the overexpression of programmed cell death protein 1 (PD-1) receptor, which decreases the anti-tumor activity of the T cells (6). More recently, the roles of the FGFR and CSF1R in inducing proliferation and differentiation of tumor-associated macrophages, thereby increasing tumor immune evasion, have been demonstrated (7).

Targeting multiple kinases to simultaneously block VEGFR-, FGFR-, and CSFIR-mediated pathways may be a more effective method of preventing tumor angiogenesis and tumor immune evasion and therefore represents an attractive approach for cancer therapy.

The compound of Formula A (“Compound A” and “compound of formula A” are used interchangeably herein), i.e., N-(2-(dimethylamino) ethyl)-1-(3-((4-((2-methyl-1H-indol-5-yl)oxy)pyrimidin-2-yl)amino)phenyl)-methanesulfonamide, and/or a pharmaceutically acceptable salt thereof was disclosed in U.S. patent application Ser. No. 13/510,249 (the '249 application), which is a national stage of PCT/CN2010/078997, filed Nov. 23, 2010, now issued as U.S. Pat. No. 8,658,658 (the '658 patent). The '658 patent is incorporated herein by reference in its entirety.

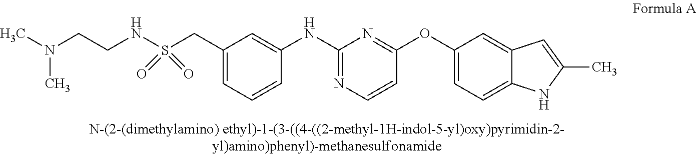

Compound A is a selective and potent small molecule, tyrosine kinase inhibitor of VEGFR 1, 2, and 3. FGFR 1, and CSFIR (8), that has demonstrated selectivity in a broad kinase screening (Supplementary Table 1). The aims of this first-in-human study were to determine the maximum tolerated dose (MTD) and recommended dose for further phase 2 investigations of compound A in patients with advanced solid tumors. In addition, the study was designed to investigate the safety, pharmacokinetics (PK), and tumor response of compound A.

SUPPLEMENTARY TABLE 1
Compound A kinase selectivity profile

| Kinase | IC₅₀ (μM) |
|---|---|
| VEGFR 1 | 0.002 |
| VEGFR 2 | 0.024 |
| VEGFR 3 | 0.001 |
| FGFR1 | 0.015 |
| CSF1R | 0.004 |
| TrkB | 0.041 |
| FLT3 | 0.067 |
| 278 other kinases | >0.150 |

CSF1R, colony stimulating factor 1 receptor;
FGFR, fibroblast growth factor receptor;
FLT3, fms-related tyrosine kinase 3;
IC₅₀, half maximal inhibitory concentration;
TrkB, tropomyosin receptor kinase B;
VEGFR, vascular endothelial growth factor receptor.

In a first aspect, provided is a method of treating a solid tumor in a patient comprising administration to the patient in need thereof a therapeutically effective amount of a compound that is an inhibitor of VEGFR 1, 2, and 3, FGFR 1, and CSF1R. In some embodiments, the compound is compound A. In some embodiments, the solid tumor is neuroendocrine tumor (NET).

In a second aspect, provided is a method of treating a solid tumor in a patient comprising administration to the patient in need thereof once daily compound A in an amount ranging from 200 to 350 mg. In some embodiments, the once daily amount of compound A is 200, 300, or 350 mg. In some embodiments, the solid tumor is NET. In some embodiments, the once daily compound A is in an amount of 300 mg. In some embodiments, the once daily compound A is in an amount of 350 mg. In some embodiments, the method demonstrates ORR (objective response rate) of greater than 20.0% and DCR (disease control rate) of greater than 65.0%. In some embodiments, the method demonstrates an ORR (objective response rate) of greater than 20.0%, DCR (disease control rate) of greater than 60.0%. In some embodiments, the method demonstrates an ORR (objective response rate) of greater than 30.0%, DCR (disease control rate) of greater than 70.0%. In some embodiments, the method demonstrates an ORR (objective response rate) of greater than 30.0%, DCR (disease control rate) of greater than 80.0%, and a median of PFS (progression-free survival) of greater than 15.0 months (95% CI: 10.3-NR).

In a third aspect, provided is a method of treating NET in a patient comprising administration to the patient in need thereof once daily compound A in an amount of 300 mg.

In a fourth aspect, provided is a method of treating NET in a patient comprising administration to the patient in need thereof once daily compound A in an amount ranging from 200 to 300 mg, wherein the method demonstrates an ORR (objective response rate) of greater than 30.0%, DCR (disease control rate) of greater than 80.0%, and a median of PFS (progression-free survival) of greater than 15.0 months (95% CI: 10.3-NR).

In a fifth aspect, provided is a method of treating a solid tumor in a patient comprising administration to the patient in need thereof once daily compound A in an amount of 300 mg, wherein the method demonstrates an ORR (objective response rate) of greater than 20.0%, and a DCR (disease control rate) of greater than 60.0%.

In a sixth aspect, provided is a method of treating a solid tumor in a patient comprising administration to the patient in need thereof once daily compound A in an amount of 350 mg, wherein the method demonstrates an ORR (objective response rate) of greater than 30.0%, and a DCR (disease control rate) of greater than 70.0%.

For each of the above aspects: in some embodiments, compound A is administered in the form of a pharmaceutical composition comprising 5, 25, 50, or 200 mg compound A; in some embodiments, compound A is administered in the form of a pharmaceutical composition comprising 50 or 200 mg compound A; in some embodiments, compound A is administered in the form of a capsule comprising 5, 25, 50, or 200 mg compound A; in some embodiments, compound A is administered in the form of a capsule comprising 50 or 200 mg compound A; in some embodiments, the capsule comprises Form I compound A (see U.S. Pat. No. 8,658,658 B2). In some embodiments, Form I compound A is micronized with a D90 value less than or equal to 11. 0 μM. In some embodiments, Form I compound A is micronized with a D90 value ranging from 3.0 to 11.0 μM.

As used herein, the term “D90 value” refers to 90% (by numbers) of the particle sizes is less than or equal to the value. For example, D90=3.5 μM means 90% (by numbers) of the particle sizes is less than or equal to 3.5 μM; D90=10.8 μM means 90% (by numbers) of the particle sizes is less than or equal to 10.8 μM.

### BRIEF DESCRIPTION OF DRAWING

FIG. 1 describes the phase 1 study design. ^{a}Compound A dose was escalated (until the MTD was met) according to a modified Fibonacci 3+3 protocol, if at least 3 evaluable patients successfully completed the DLT observation period according to the criteria described in the “Materials and Methods” section. Protocol planned for dose escalation to 400 mg QD; however, the drug exposure (AUC, Cₘₐₓ) at dose of 350 mg QD did not increase vs 300 mg QD. Based on the available PK, safety, and efficacy data, the investigator and sponsor agreed there would be no further dose escalation to 400 mg QD or above even though MTD had not been reached. Each patient was allocated a dose and received that dose for the duration of the study. ^{b}The tumor expansion phase was initiated following determination of the recommended phase 2 dose based on the results of the dose-escalation phase. AUC, area under the curve; BID, twice daily; Cₘₐₓ, peak concentration; DLT, dose limiting toxicity, F, formulation; MTD, maximum tolerated dose; PK, pharmacokinetics; QD, once daily.

FIG. 2 describes best percent change in tumor size (sum of diameter of target lesions) compared with baseline for efficacy-evaluable patients treated with compound A formulation 2 (N=28). GI, gastrointestinal; HCC, hepatocellular carcinoma; NET, neuroendocrine tumors; PD, progressive disease; PNET, pancreatic neuroendocrine tumor; PR, partial response; SD, stable disease; VEGFR, vascular endothelial growth factor receptor.

FIG. 3 describes Kaplan-Meier survival curve of PFS in patients with NET treated with compound A formulation 2 (N=21). NET, neuroendocrine tumors; PFS, progression-free survival.

FIG. 4 describes patient disposition. ^{a}At enrollment, patients were allocated a dose sequentially according to the Fibonacci 3+3 dose-escalation design. Patients continued to receive that dose until study discontinuation. ^{b}Patients who completed the DLT observation phase could remain on treatment at their original dose until disease progression or any other withdrawal criteria were met. BID, twice daily; DLT, dose limiting toxicity; QD, once daily

### EXPERIMENTS

### I. CSF1R Inhibition

Inhibition of compound A on Fins was determined using [³²p-ATP] incorporation assay carried by Eurofins Pharma Discovery Services UK Ltd. Fms kinase was incubated with 8 mM MOPS (3-(N-Morpholino)propanesulfonic acid sodium) pH 7.0, 0.2 mM EDTA ((Ethylenedinitrilo)tetraacetic acid disodium salt), 250 μM KKKSPGEYVNIEFG (a peptide), 10 mM MgAcetate and [γ-33P-ATP] (specific activity approx. 500 cpm/pmol, concentration as required). The reaction was initiated by the addition of the MgATP mix. After incubation for 40 minutes at room temperature, the reaction was stopped by the addition of 3% phosphoric acid solution. 10 μL of the reaction was then spotted onto a P30 filtermat and washed three times for 5 minutes in 75 mM phosphoric acid and once in methanol prior to drying and scintillation counting.

Results: IC₅₀ of compound A on Fms kinase was determined as 0.004 μM.

| Concentration | | |
|---|---|---|
| (μM) | IH (%) | IC₅₀ (μM) |
| 1 | 98 | 0.004 |
| 0.3 | 98 | |
| 0.1 | 94 | |
| 0.03 | 86 | |
| 0.01 | 72 | |
| 0.003 | 38 | |
| 0.001 | 15 | |
| 0.0003 | 3 | |
| 0.0001 | −3 | |

### II. Phase 1 Clinical Study

### Summary

This phase 1 study (NCT02133157) assessed the maximum tolerated dose (MTD), recommended phase 2 dose, safety, and pharmacokinetics of compound A in patients with advanced solid tumors.

The study consisted of a dose-escalation phase (50-350 mg/day, 28-day cycle) with a Fibonacci (3+3) design investigating MTD, phase 2 dose, dose-limiting toxicities (DLTs), and pharmacokinetics; and a tumor-specific expansion phase investigating the tumor response (RECIST V1.0 criteria) to the identified compound A dose. Safety was assessed throughout. Two formulations were assessed: formulation 1 (5 mg, 25 mg, and 50 mg capsules) and formulation 2 (50 mg and 200 mg capsules). The excipients contained in formulation 1 and formulation 2 are substantially similar. Compound A in formulation 1 is not micronized whereas compound A in formulation 2 is micronized.

Formulation 2

Formulation 2

| | 200 mg capsule^{a} | 50 mg capsule^{b} |
|---|---|---|
| Compound A, Form I | 200 mg | 50 mg |
| Microcrystalline Cellulous | 178 mg | 79.35 mg |
| Mannitol | — | — |
| Sodium Starch Glycolate | 9 mg | — |
| Silicon dioxide | 9 mg | — |
| Magnesium stearate | 4 mg | 0.65 mg |

^{a}200 mg capsule comprises micronized compound A Form I with D90 value as 8.1, 9.6, or 10.8 μM.
^{b}50 mg capsule comprises micronized compound A Form I with D90 value as 3.5, 8.1, 9.6, or 10.8 μM.

Seventy-seven Chinese patients received oral compound A. Compound A exposure generally increased proportionally to dose from 50-300 mg, and plateaued above 300 mg; MTD was not reached. DLTs (dose-limiting toxicity) included abnormal hepatic function and coagulation tests and upper gastrointestinal hemorrhage. The most common treatment-related adverse events (AEs) were proteinuria, hypertension, and diarrhea. Among the 34 patients receiving compound A formulation 2, one patient with hepatocellular carcinoma and eight patients with neuroendocrine tumors (NETs) achieved partial response; 15 patients had stable disease. The objective response rate was 26.5% (9/34) and the disease control rate was 70.6% (24/34).

### Materials and Methods

### Patients

Patients were recruited from the 307th Hospital of Chinese People's Liberation Army, Beijing, China, and Peking University Cancer Hospital, Beijing, China. Patients with recurrent and/or metastatic malignant solid tumors who had shown disease progression after treatment standard therapy or were unable to receive standard therapy or for whom there was no standard therapy were eligible for this study. Eligible patients were 18-75 years old, with an Eastern Cooperative Oncology Group (ECOG) performance status ≤1, and a life expectancy of more than 3 months. Patients with uncontrolled brain metastases were excluded. Pre-treatment evaluations included: a physical examination; ECOG performance status; laboratory tests for renal, liver, and metabolic functions; cardiac function (electrocardiogram and ultrasonic cardiogram); and a pregnancy test for female patients of childbearing age.

### Study Design and Dose Administration

The primary objectives of this open-label, first-in-human phase 1 study (NCT02133157) were to determine MTD and the phase 2 dose of compound A, and to evaluate the safety of compound A in patients with advanced solid tumors. The secondary and exploratory objectives included evaluation of PK and preliminary anti-tumor activity of compound A.

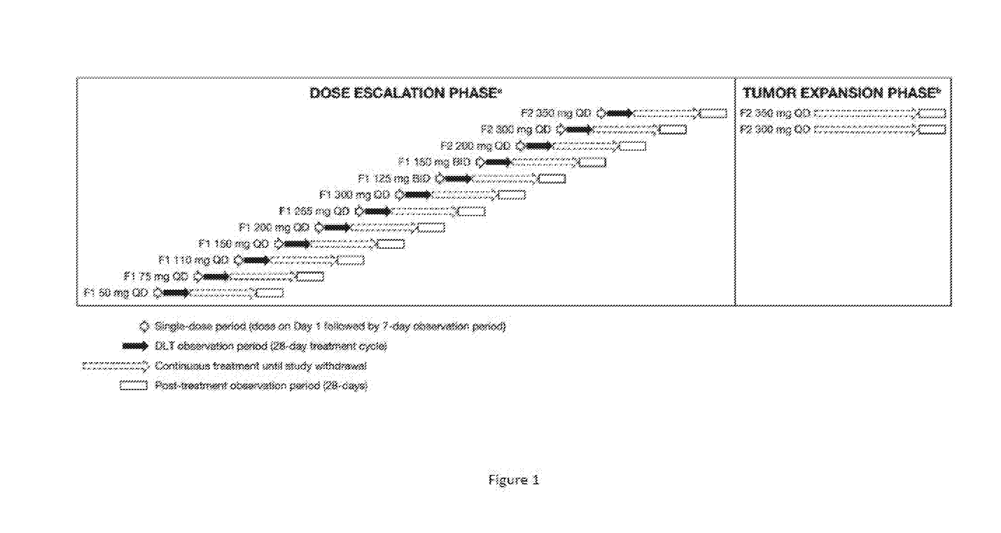

The study consisted of a dose-escalation phase (split into a single-dose period and continuous-dose period) and a tumor-specific expansion phase (FIG. 1). Two formulations of compound A were used during the study formulation—1 (5, 25, and 50 mg capsules) and formulation 2 (50 and 200 mg capsules).

The study was conducted in accordance with the Good Clinical Practice Guidelines of the International Council for Harmonisation of Technical Requirements for Pharmaceuticals for Human Use. The protocol was approved by each participating institution's ethics review board. All patients were required to provide written informed consent.

### Dose-Escalation Phase

During the dose-escalation phase, patients were given a single dose of compound A and monitored for adverse events (AEs) for 7 days. If no clinically significant toxicities were observed, patients could enter the 28-day dose-limiting toxicity (DLT) observation phase where they received compound A continuously for 28 days. DLTs were assessed at the end of the 28-day period. If no patients experienced a DLT during the 28-day period, the dose was escalated (FIG. 1). After the completion of the DLT observation phase, patients could continue compound A treatment at their current dose (if they were judged by the investigator to be benefiting from the treatment) until any of the withdrawal criteria (investigator's decision that withdrawal was in the patient's best interest, intolerable toxicity, disease progression) were met.

The study used a modified Fibonacci 3+3 dose-escalation design with at least three evaluable patients treated with each dose. In total, 12 dose cohorts were assessed (FIG. 1). The MTD was defined as the maximum dose at which no more than one of six evaluable patients experienced a DLT during the first 28-day treatment period (cycle). For each dose, if no patients experienced a DLT during the treatment cycle, the dose was escalated for the next dose cohort. If one of the first three patients treated at a dose experienced a DLT, three additional patients were added to expand the cohort. If one of six patients experienced a DLT, the MTD was considered exceeded and the previous lower dose would be re-assessed to determine the MTD. Patients who completed the first treatment cycle and fulfilled ≥75% of the planned accumulating dose, or who experienced a DLT any time during the first treatment cycle, were considered as DLT-evaluable patients. Dose reductions were not permitted in the first treatment cycle.

### Tumor-Specific Expansion Phase

After the MTD/recommended phase 2 dose was established and preliminary efficacy data from the dose-escalation phase had demonstrated an effective dose range (a partial response [PR] had been observed), the study was expanded to investigate the tumor response at the identified doses (300 mg and 350 mg once daily {QD} [formulation 2]) in patients with advanced solid tumors. Patients with neuroendocrine tumors (NETs) were enrolled with priority as preliminary efficacy was shown in these tumor types in the dose-escalation stage.

### Endpoints and Analyses

### Toxicity and DLT Assessments

Safety and tolerability were assessed in all patients who received at least one dose of study medication throughout the study. AEs were recorded throughout the study. All AEs were coded by organ system using preferred terms as per the Medical Dictionary for Regulatory Activities (MedDRA) Version 17.0. All AEs were graded using the National Cancer Institute Common Terminology Criteria for Adverse Events (NCI CTCAE) Version 3.0. AE frequency, type, severity, and relationship to study drug were summarized and tabulated, together with the incidence of serious AEs (SAEs) or deaths. Treatment-related AEs (TRAEs) were defined as AEs that were considered by the investigators to be possibly, probably, or definitely related to the study treatment.

DLTs were defined as any of the following toxicities occurring in the first continuous-dosing treatment cycle (Day 1-28) of the dose-escalation phase: any non-hematologic toxicity ≥Grade 3 in severity, except for fatigue, nausea, vomiting, diarrhea, constipation, pain, and hypertension which were considered DLTs if they were ≥Grade 3 severity after adequate treatment; hematologic toxicities, including Grade 4 decreases in white blood cell count, platelet count or hemoglobin; Grade 3 febrile neutropenia; and Grade 3 decreases in platelets with hemorrhage tendency.

Physical examinations, ECOG performance status, and laboratory tests were obtained in the single-dose screening period at Day 1, and during the continuous-dose period were collected weekly in the first treatment cycle, every two weeks in the second treatment cycle, and every four weeks for treatment cycle 3 onwards.

### PK Assessments

For the assessment of compound A PK at steady state, plasma samples were collected from each patient at pre-dose, 1, 2, 4, 8, 12, and 24 hours on Day 14 for QD cohorts or at pre-dose, 1, 4, 8, and 12 hours following the first dose on Day 14 for twice daily (BID) cohorts.

PK parameters analyzed included area under the concentration-time curve (AUC), peak concentration (Cₘₐₓ), and time to Cₘₐₓ (Tₘₐₓ). Phoenix WinNonlin 6.3 software was used to analyze descriptive statistics of concentration data and PK parameters and to plot plasma concentration-time curves. AUC was calculated using the linear trapezoidal area method.

### Clinical Response

Tumor response (an exploratory endpoint) was assessed according to Response Evaluation Criteria in Solid Tumors (RECIST) Version 1.0 and measured at baseline, and at the end of every treatment cycle in the first 4 cycles and every other cycle thereafter. Patients with an initial assessment of complete response (CR) or partial response (PR) had to have the result confirmed by a repeat tumor assessment at least 4 weeks later. Calculations of the following parameters were made: objective response rate (ORR) (CR+PR); stable disease (SD) defined as ≥1 assessment of SD at least 6 weeks after study entry; disease control rate (DCR) (CR+PR+SD). Time to response (TTR), duration of response (DoR), and progression-free survival (PFS) were assessed in the subgroup of patients with NET.

### Results

### Patient Baseline Characteristics

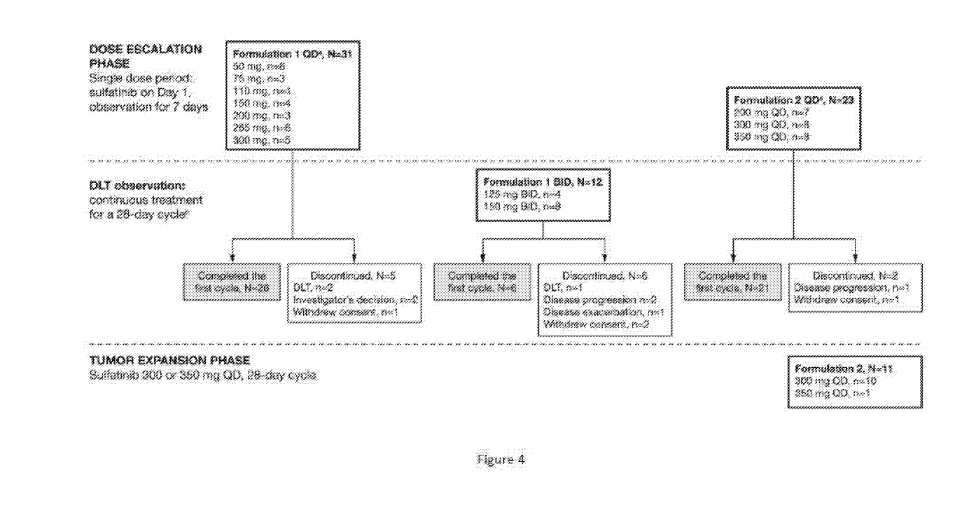

Seventy-seven Chinese patients were enrolled between April 2010 and September 2014 and followed up until July 2015. The first 43 patients received compound A formulation 1 and the remaining 34 patients received compound A formulation 2 (FIG. 4). Baseline demographic and clinical characteristics for patients treated with formulations 1 and 2 are summarized in Table 1.

TABLE 1
Baseline demographic and clinical characteristics

| | Formulation 1 | | Formulation 2 | |
|---|---|---|---|---|
| Characteristic | (N = 43) | | (N = 34) | |
| Median (range) age, years | 52.7 | (23.5-69.9) | 56.0 | (23.4-73.2) |
| Gender, n (%) | | | | |
| Male | 27 | (62.8) | 24 | (70.6) |
| Female | 16 | (37.2) | 10 | (29.4) |
| Tumor type, n (%) | | | | |
| Colorectal carcinoma | 9 | (20.9) | 0 | |
| Hepatocellular carcinoma | 8 | (18.6) | 9 | (26.5) |
| Stromal tumor | 8 | (18.6) | 1 | (2.9) |
| NET (G1/G2)^{a} | 7 | (16.3) | 21 | (61.8) |
| Non-small cell lung cancer | 2 | (4.7) | 0 | |
| Renal cell carcinoma | 2 | (4.7) | 0 | |
| Other | 7 | (16.3) | 3 | (8.8) |
| ECOG performance status, | | | | |
| n (%) | | | | |
| 0 | 10 | (23.3) | 4 | (11.8) |
| 1 | 29 | (67.4) | 30 | (88.2) |
| 2 | 4 | (9.3) | 0 | |
| Median (range) time since | 1.9 | (0.1-11.2) | 0.8 | (0.0-6.8) |
| diagnosis, years | | | | |
| Previous anti-tumor systemic | | | | |
| therapy, n (%) | | | | |
| Yes | 33 | (76.7) | 20 | (58.8) |
| No | 10 | (23.3) | 14 | (41.2) |

^{a}The pathology grading of neuroendocrine tumors was categorized according to Ki67 index and mitotic rate of the tumor cells. G1 and G2 tumors were also reported as well-differentiated NET.
ECOG, Eastern Cooperative Oncology Group;
G1, low grade NET;
G2, intermediate grade NET;
NET, neuroendocrine tumor.

### Compound a Exposure, Dose Escalation, and DLTs

Sixty-six patients were enrolled in the dose-escalation phase; of these, 53 (80.3%) completed the first treatment cycle. The reasons for discontinuation were disease progression (n=3) or disease deterioration (n=1; total n=4, 6.1%), consent withdrawal (n=4, 6.1%), DLTs (n=3, 4.5%), and investigator's decision (n=2, 3.0%).

In the dose-escalation phase, 43 patients received continuous treatment with compound A formulation 1 at doses of 50 mg, 75 mg, 110 mg, 150 mg, 200 mg, 265 mg, and 300 mg QD, and 125 mg and 150 mg BID (Supplementary FIG. 1). The median treatment duration with formulation 1 was 32.5 days (range 2-269 days). Three patients experienced DLTs (one Grade 3 abnormal coagulation with compound A 50 mg QD; one Grade 3 upper gastrointestinal [GI] hemorrhage with compound A 265 mg QD; one Grade 3 abnormal hepatic function with compound A 150 mg BID).

Twenty-three patients received compound A formulation 2 during the dose-escalation phase at doses of 200 mg, 300 mg, and 350 mg QD (Supplementary FIG. 1). In addition, there were 11 patients treated with 300 mg or 350 mg QD compound A formulation 2 during the expansion phase. The median treatment duration with formulation 2 was 147.5 days (range 9-644 days). One patient receiving compound A formulation 2 at 200 mg QD experienced a DLT (Grade 3 abnormal hepatic function).

MTD was not reached with compound A doses of up to 350 mg QD (formulation 2). The initial plan was to escalate the dose of formulation 2 up to 400 mg QD; however, the drug exposure (AUC, Cₘₐₓ) at a dose of 350 mg QD was no higher than that with 300 mg QD. Based on the available PK, safety, and efficacy data, the investigator and sponsor agreed there would be no further dose escalation although MTD had not been reached.

### Safety

### Formulation 1

Forty-two patients (97.7%) receiving formulation 1 experienced one or more AEs. The most common TRAEs (occurring in ≥10% patients) were: asthenia; increased blood bilirubin; proteinuria; increased aspartate aminotransferase (AST); diarrhea; increased blood pressure; hypomagnesemia; increased white blood cell count; abdominal pain; hematuria; hypocalcemia; hypokalemia; and pyrexia.

The incidence of Grade 3 TRAEs was 25.6%, of which the most common were increased AST (n=2, 4.7%) and decreased hemoglobin (n=2, 4.7%). The incidence rates of all other Grade 3 TRAEs (abdominal pain, diarrhea, upper GI hemorrhage, gastric dysfunction, abnormal hepatic function, gastroenteritis, infection, increased alanine transaminase (ALT), increased blood pressure, abnormal coagulation test, hypokalemia, hypoproteinemia, nephrotic syndrome, and pelvi-ureteric obstruction) were all 2.3% (n=1).

There were no Grade 4 or Grade 5 TRAEs. There were five SAEs, three of which were considered by the investigator as possibly related to the study drug: a Grade 3 nephrotic syndrome and a Grade 3 upper GI hemorrhage (both in patients receiving 265 mg QD); and a Grade 3 hepatic function abnormality in a patient receiving 150 mg BID. These three patients discontinued compound A and received supportive care. The patient with a Grade 3 hepatic function abnormality died 23 days after the last study dose; disease progression was considered the primary cause of death by the investigator.

### Formulation 2

All patients treated with formulation 2 experienced at least one AE. The most commonly reported TRAEs of any grade (occurring in ≥10% of patients) are summarized in Table 2. The overall incidence of Grade 3 and Grade 4 TRAEs was 47.1%, the most common was proteinuria (14.7%; Table 2). No Grade 5 AEs were reported.

TABLE 2
TRAEs occurring in ≥10% of patients treated with compound A formulation 2 (N = 34)

| System organ class | 200 mg QD, N = 7 | | 300 mg QD, N = 18 | | 350 mg QD, N = 9 | | Total, N = 34 | |
|---|---|---|---|---|---|---|---|---|
| Preferred term, | Any | Grade | Any | Grade | Any | Grade | Any | Grade |
| n (%) | grade | 3/4 | grade | 3/4 | grade | 3/4 | grade | 3/4 |

Blood and lymphatic system
disorders

| Sinus bradycardia | 0 | 0 | 4 | (22.2) | 0 | 1 (11.1) | 0 | 5 | (14.7) | 0 |
|---|---|---|---|---|---|---|---|---|---|---|
| Anemia | 0 | 0 | 2 | (11.1) | 0 | 2 (22.2) | 1 (11.1) | 4 | (11.8) | 1 (2.9) |
| Gastrointestinal disorders | | | | | | | | | | |
| Diarrhea | 3 (42.9) | 0 | 8 | (44.4) | 1 (5.6) | 8 (88.9) | 1 (11.1) | 19 | (55.9) | 2 (5.9) |
| Abdominal discomfort | 2 (28.6) | 0 | 3 | (16.7) | 0 | 6 (66.7) | 0 | 11 | (32.4) | 0 |
| Nausea | 3 (42.9) | 0 | 4 | (22.2) | 0 | 2 (22.2) | 0 | 9 | (26.5) | 0 |
| Abdominal distention | 1 (14.3) | 0 | 4 | (22.2) | 0 | 4 (44.4) | 0 | 9 | (26.5) | 0 |
| General disorders and | | | | | | | | | | |
| administration site | | | | | | | | | | |
| conditions | | | | | | | | | | |
| Asthenia | 2 (28.6) | 0 | 4 | (22.2) | 0 | 6 (66.7) | 1 (11.1) | 12 | (35.3) | 1 (2.9) |
| Face edema | 0 | 0 | 2 | (11.1) | 1 (5.6) | 5 (55.6) | 0 | 7 | (20.6) | 1 (2.9) |
| Edema peripheral | 0 | 0 | 2 | (11.1) | 1 (5.6) | 3 (33.3) | 0 | 5 | (14.7) | 1 (2.9) |
| Investigations | | | | | | | | | | |
| Blood pressure | 0 | 0 | 10 | (55.6) | 2 (11.1) | 4 (44.4) | 0 | 14 | (41.2) | 2 (5.9) |
| increased | | | | | | | | | | |
| Blood TSH increased | 1 (14.3) | 0 | 7 | (38.9) | 0 | 5 (55.6) | 0 | 13 | (38.2) | 0 |
| Blood bilirubin | 2 (28.6) | 0 | 8 | (44.4) | 1 (5.6) | 3 (33.3) | 0 | 13 | (38.2) | 1 (2.9) |
| increased | | | | | | | | | | |
| AST increased | 2 (28.6) | 1 (14.3) | 6 | (33.3) | 1 (5.6) | 4 (44.4) | 0 | 12 | (35.3) | 2 (5.9) |
| Blood triglycerides | 0 | 0 | 5 | (27.8) | 0 | 7 (77.8) | 0 | 12 | (35.3) | 0 |
| increased | | | | | | | | | | |
| WBC count decreased | 1 (14.3) | 0 | 5 | (27.8) | 0 | 5 (55.6) | 0 | 11 | (32.4) | 0 |
| Neutrophil count | 2 (28.6) | 0 | 4 | (22.2) | 1 (5.6) | 4 (44.4) | 0 | 10 | (29.4) | 1 (2.9) |
| decreased | | | | | | | | | | |
| Blood albumin | 1 (14.3) | 0 | 8 | (44.4) | 0 | 1 (11.1) | 0 | 10 | (29.4) | 0 |
| decreased | | | | | | | | | | |
| Platelet count | 2 (28.6) | 0 | 3 | (16.7) | 0 | 5 (55.6) | 2 (22.2) | 10 | (29.4) | 2 (5.9) |
| decreased | | | | | | | | | | |
| Abnormal ECG | 1 (14.3) | 0 | 6 | (33.3) | 0 | 2 (22.2) | 0 | 9 | (26.5) | 0 |
| T-wave | | | | | | | | | | |
| Blood uric acid | 1 (14.3) | 0 | 4 | (22.2) | 1 (5.6) | 4 (44.4) | 0 | 9 | (26.5) | 1 (2.9) |
| increased | | | | | | | | | | |
| Blood triglyceride | 0 | 0 | 4 | (22.2) | 0 | 4 (44.4) | 0 | 8 | (23.5) | 0 |
| increased | | | | | | | | | | |
| Hyperuricemia | 0 | 0 | 4 | (22.2) | 1 (5.6) | 3 (33.3) | 0 | 7 | (20.6) | 1 (2.9) |
| ALT increased | 2 (28.6) | 1 (14.3) | 2 | (11.1) | 0 | 2 (22.2) | 0 | 6 | (17.6) | 1 (2.9) |
| Blood creatinine | 1 (14.3) | 0 | 0 | | 0 | 5 (55.6) | 0 | 6 | (17.6) | 0 |
| increased | | | | | | | | | | |
| Thyroxine free | 0 | 0 | 5 | (27.8) | 0 | 1 (11.1) | 0 | 6 | (17.6) | 0 |
| decreased | | | | | | | | | | |
| Hemoglobin decreased | 1 (14.3) | 0 | 1 | (5.6) | 1 (5.6) | 3 (33.3) | 1 (11.1) | 5 | (14.7) | 2 (5.9) |
| Thyroid function test | 1 (14.3) | 0 | 1 | (5.6) | 0 | 2 (22.2) | 0 | 4 | (11.8) | 0 |
| abnormal | | | | | | | | | | |
| Metabolism and nutrition | | | | | | | | | | |
| disorders | | | | | | | | | | |
| Hypoproteinemia | 0 | 0 | 10 | (55.6) | 0 | 7 (77.8) | 0 | 17 | (50.0) | 0 |
| Hypocalcemia | 0 | 0 | 7 | (38.9) | 0 | 5 (55.6) | 0 | 12 | (35.3) | 0 |
| Decreased appetite | 0 | 0 | 6 | (33.3) | 0 | 5 (55.6) | 0 | 11 | (32.4) | 0 |
| Hypokalemia | 2 (28.6) | 0 | 4 | (22.2) | 0 | 2 (22.2) | 1 (11.1) | 8 | (23.5) | 1 (2.9) |
| Hypophosphatemia | 2 (28.6) | 0 | 3 | (16.7) | 2 (11.1) | 1 (11.1) | 0 | 6 | (17.6) | 2 (5.9) |
| Hyponatremia | 0 | 0 | 3 | (16.7) | 0 | 1 (11.1) | 0 | 4 | (11.8) | 0 |
| Hypercholesterolemia | 0 | 0 | 0 | | 0 | 4 (44.4) | 0 | 4 | (11.8) | 0 |
| Musculoskeletal and | | | | | | | | | | |
| connective tissue disorders | | | | | | | | | | |
| Dizziness | 1 (14.3) | 0 | 2 | (11.1) | 0 | 3 (33.3) | 0 | 6 | (17.6) | 0 |
| Back pain | 1 (14.3) | 0 | 3 | (16.7) | 0 | 1 (11.1) | 0 | 5 | (14.7) | 0 |
| Renal and urinary disorders | | | | | | | | | | |
| Proteinuria | 2 (28.6) | 0 | 11 | (61.1) | 3 (16.7) | 7 (77.8) | 2 (22.2) | 20 | (58.8) | 5 (14.7) |
| Vascular disorders | | | | | | | | | | |
| Hypertension | 0 | 0 | 5 | (27.8) | 1 (5.6) | 2 (22.2) | 0 | 7 | (20.6) | 1 (2.9) |

Eight SAEs were reported, two of which were considered by the investigator to be possibly related to the study drug: a Grade 3 upper GI hemorrhage in the 300 mg QD dose cohort; and a case of Grade 3 acute pancreatitis in the 350 mg QD dose cohort. The majority of SAEs were resolved with the exception of an intra-abdominal hemorrhage (unrelated to the study drug) and an intervertebral disc protrusion (unlikely related to the study drug).

### Pharmacokinetic Profile

Sixty-eight patients were eligible for the steady-state PK assessment, including 40 patients who received compound A formulation 1 and 28 patients who received formulation 2 (Table 3).

TABLE 3
Compound A pharmacokinetic parameters on Day 14 of continuous dosing

| | Compound A formulation 1 dose | | | | | | | | |
|---|---|---|---|---|---|---|---|---|---|
| PK | 50 mg QD | 75 mg QD | 110 mg QD | 150 mg QD | 200 mg QD | 265 mg QD | 300 mg QD | 125 mg BID | 150 mg BID |
| parameter | (N = 5) | (N = 3) | (N = 4) | (N = 3) | (N = 3) | (N = 6) | (N = 5) | (N = 3) | (N = 8) |
| Mean Cₘₐₓ | 83.7 (56.4) | 123 (69.5) | 262 (35.3) | 293 (35.7) | 370 (20.3) | 498 (66.9) | 546 (67.3) | 267 (52.1) | 248 (65.9) |
| (CV %), | | | | | | | | | |
| ng/mL | | | | | | | | | |
| Median | 1.8 | 2.0 | 1.5 | 1.0 | 3 | 3.5 | 2.8 | 3.0 | 3.4 |
| Tₘₐₓ | (1.0-4.0) | (1.0-4.0) | (0-4.0) | (1.0-1.0) | (1.0-4.0) | (1.0-4.0) | (1.0-4.0) | (1.0-4.0) | (1.0-8.0) |
| (range), | | | | | | | | | |
| hour | | | | | | | | | |
| Mean | 654 (51.2) | 964 (32.3) | 2579 (28.2) | 2308 (48.8) | 3314 (13.1) | 5958 (65.2) | 5403 (68.8) | 1977 (59.8) | 1952 (64.8) |
| AUC^{a} | | | | | | | | | |
| (CV %), | | | | | | | | | |
| ng · hour/mL | | | | | | | | | |

| | Compound A formulation 2 dose | | |
|---|---|---|---|
| PK | 200 mg QD | 300 mg QD | 350 mg QD |
| parameter | (N = 6) | (N = 14) | (N = 8) |
| Mean Cₘₐₓ | 549 (73.1) | 625 (54.6) | 655 (32.5) |
| (CV %), | | | |
| ng/mL | | | |
| Median | 1.0 | 2.0 | 2.0 |
| Tₘₐₓ | (1.0-2.0) | (1.0-4.1) | (2.0-4.0) |
| (range), | | | |
| hour | | | |
| Mean | 4273 (55.0) | 5116 (50.4) | 5289 (37.6) |
| AUC^{a} | | | |
| (CV %), | | | |
| ng · hour/mL | | | |

^{a}AUC₀₋₂₄ for QD and AUC₀₋₁₂ for BID.
AUC, area under the curve; BID, twice daily; Cₘₐₓ, peak concentration; CV %, coefficient of variation; PK, pharmacokinetic; QD, once daily; Tₘₐₓ, time to Cₘₐₓ.

### Formulation 1

Following consecutive QD administration for 14 days, within the dose range of 50 mg to 265 mg, compound A exposure (indicated by AUC) generally increased dose-proportionally. There was no AUC increase when the dose increased from 265 mg to 300 mg. The median Tₘₐₓ ranged from 1.8 to 3.5 hours. Both Cₘₐₓ and AUC showed high inter-subject variability with CV % of up to 69.5% for Cₘₐₓ (75 mg group) and 68.8% for AUC (300 mg group). Following BID administration for 14 days, the mean AUC values were similar at 125 and 150 mg (1977 vs 1952 ng-hour/mL) with the CV % up to 64.8%.

### Formulation 2

Following consecutive QD administration for 14 days, mean AUC at 200, 300, and 350 mg was 4273, 5116, and 5289 ng·hour/mL, respectively, which indicated that 300 mg and 350 mg were similar but higher than 200 mg in terms of compound A exposure. The inter-subject variability was high, as indicated by the CV % of up to 55% for AUC and 73.1% for Cₘₐₓ. Median Tₘₐₓ ranged from 1.0 to 2.0 hours for the test dose levels.

### Clinical Response

Among 43 patients treated with compound A formulation 1, 12 patients were not evaluable for efficacy either because they did not have a post-treatment tumor assessment, or because they had SD at the first post-treatment assessment (Week 4) but no additional assessment to demonstrate that the SD continued for a minimum of 6 weeks from baseline. Of the 31 patients evaluable by RECIST criteria, no patients achieved a CR or PR; eight patients had SD and 23 had progressive disease (PD).

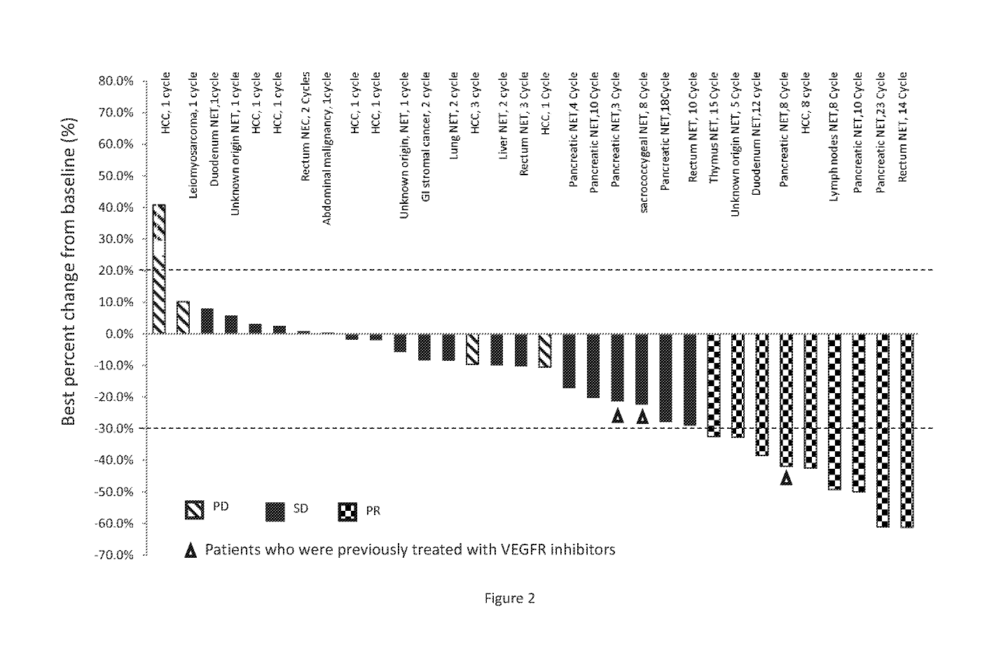

Among the 34 patients treated with formulation 2, six patients were not evaluable for response due to early discontinuation in cycle 1 (Supplementary FIG. 1). Of the 28 efficacy evaluable patients by RECIST criteria, nine patients achieved a PR (FIG. 2); one patient with hepatocellular carcinoma receiving compound A 200 mg QD and eight with NET receiving compound A 300 or 350 mg QD. There were 15 patients with SD (10 patients with NET, three with hepatocellular carcinoma, one with GI stromal tumors, and one patient with an abdominal malignancy) and four patients with PD.

Among all 77 patients the ORR was 11.7% (9/77) and DCR was 41.6% (32/77). The ORR of patients treated with compound A formulation 2 was 26.5% (9/34) and DCR was 70.6% (24/34).

Tumor response was similar among patients receiving the highest doses of formulation 2 (300 mg QD: ORR 27.8%. DCR 66.7% [n=18]; 350 mg QD: ORR 33.3%, DCR 77.8%) [n=9]).

There were 21 patients with NET treated with compound A formulation 2 across 200-350 mg QD dosing. Among this subgroup the ORR was 38.1% (8/21) and the DCR was 85.7% (18/21); eight patients achieved a PR, 10 patients achieved SD, and three patients were not evaluable for response.

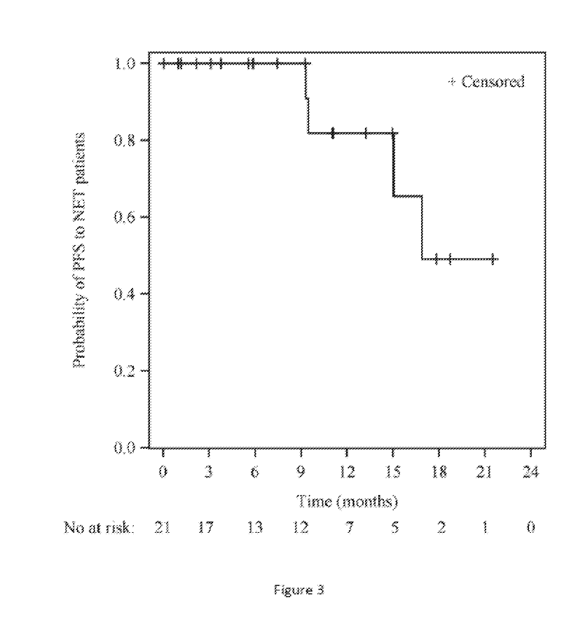

The tumor origins of the eight NET patients who achieved a PR were: pancreas (n=3); duodenum (n=1); rectum (n=1); thymus (n=1); and unknown origin (n=2). Median time to response (TTR) was 3.8 months (range 1.4-11.1 months). The median DoR was 17.0 months (95% confidence interval [CI]: 8.3—not reached [NR]). The median PFS was 18.3 months (95% CI: 10.3-NR) (FIG. 3). Notably, three NET patients who had previously been treated but progressed with VEGFR kinase inhibitors (such as sunitinib or famitinib) obtained significant clinical benefit from compound A, with two patients achieving SD and one patient achieving a PR (treatment duration from 5.5 to 12.6 months).

Compound A, a potent oral kinase inhibitor targeting VEGFR, FGFR1, and CSF1R with good selectivity over other kinases, has demonstrated anti-angiogenic and anti-tumor activity in preclinical studies (Hutchison MediPharma unpublished data). This first-in-human, phase 1 study was designed to determine the safety, PK characteristics, and anti-tumor activity of compound A in patients with advanced solid tumors.

Within compound A doses of 50-350 mg QD and 125-150 mg BID, the MTD was not reached. Compound A appeared to be generally well tolerated. Most AEs were mild to moderate and could be managed through dose adjustment or supportive care. The most commonly reported AEs, including hypertension, proteinuria, and diarrhea, were consistent with AEs seen with VEGFR tyrosine kinase inhibitors (9-12).

PK analyses demonstrated that oral compound A was rapidly absorbed and the drug exposures (AUC and Cₘₐₓ) generally increased with an increase in dose. Drug exposures started to plateau at a dose of 265 mg for formulation 1 and 300 mg for formulation 2, suggesting potential saturation in absorption. Inter-patient variation in drug exposure was found to be moderate to high across all dose levels for formulation 1, and appeared to be moderately improved for formulation 2.

Compound A demonstrated promising anti-tumor activity against advanced solid tumors; of the total 77 patients, nine patients had a confirmed PR and 23 had sustainable SD. Clinical efficacy was seen with compound A formulation 2 at doses from 200 mg QD, with nine patients achieving a PR and 15 patients reporting SD.

Unresectable or metastatic NET is a life-threatening disease with limited effective treatment options (13-15). The median survival duration varies from several months to a few years depending on the primary tumor sites (16). In recent years sunitinib, a multi-kinase inhibitor (targeting VEGFR 1, 2, 3; platelet derived growth factor receptor [PDGFR]-α and -β, kit, fins-like tyrosine kinase 3 [FLT-3], and rearranged during transfection [RET]) has been approved by the US Food and Drug Administration (FDA) for the treatment of advanced pancreatic NET. Everolimus, an oral mammalian target of rapamycin (mTOR) inhibitor, has been approved by the US FDA for the treatment of pancreatic, GI, and lung NETs. In phase 3 trials, both therapies have demonstrated ORRs of <10% (for sunatinib: 9.3% in pancreatic NET, for everolimus: 5% in pancreatic NET, and 2% in GI and lung NET) and median PFS of approximately 11 months (11, 17, 18).

In this preliminary study of efficacy in 21 patients with advanced NET treated with compound A formulation 2, robust clinical activity was demonstrated with an ORR of 38.1%, DCR of 85.7%, and a median PFS of 18.3 months (95% CI: 10.3-NR). Notably, anti-tumor activity was demonstrated by compound A in NET patients regardless of tumor origin and also in three patients who had previously failed treatment with other VEGFR inhibitors. This suggests that compound A, which targets both tumor angiogenesis and immune evasion simultaneously, may provide clinical benefit for NET patients (7, 19). Further investigations into the mechanism of action of the anti-tumor activity of compound A are ongoing, both in preclinical and clinical settings, and may provide further support for the use of compound A in advanced solid tumors.

MTD was not reached in this study. Based on the PK, safety and efficacy results, 300 mg QD was selected as recommended dose for further efficacy evaluation. Compound A was well tolerated up to 350 mg QD although the drug exposure (AUC) did not substantially alter with the dose increase from 300 mg QD to 350 mg QD (formulation 2). This finding suggested potential saturation in absorption and that further dose escalation would not achieve an increase in drug exposure. Comparing AEs in patients that received compound A 300 mg and 350 mg QD, there was a higher incidence of Grade ≥3 AEs with the 350 mg dose during the first cycle of the continuous dose period.

These two dose cohorts (formulation 2) demonstrated comparable anti-tumor activity, with ORR of 27.8% and DCR of 66.7% for patients in the 300 mg QD group (n=18) and ORR of 33.3% and DCR of 77.8% in the 350 mg QD group (n=9); however, sample sizes were small. Together, the PK, safety, and efficacy data support the selection of 300 mg QD as the recommend phase 2 dose.

The efficacy analysis should be interpreted with caution due to the small sample size, and the non-randomized, open-label study design with no comparator. Nevertheless, the preliminary results of this study provide sufficient support to warrant further anti-tumor efficacy evaluation of compound A.

In summary, compound A is an oral kinase inhibitor that targets tumor angiogenesis and immune evasion simultaneously. It has demonstrated promising anti-tumor activity in patients with advanced solid tumors, such as NETs, and was generally well tolerated. Based on the overall safety and tolerability, PK properties, and preliminary clinical efficacy, the dose selected for phase 2 clinical studies was compound A 300 mg QD. Compound A 300 mg/day demonstrated an acceptable safety profile and encouraging anti-tumor activity in patients with advanced solid tumors, such as NETs.

As a result of this study and an ongoing phase 2 study in 81 patients with NETs (NCT02267967), two randomized, double-blind, placebo-controlled, multicenter phase 3 trials are currently in progress in China—one in patients with pancreatic NETs (NCT02589821) and one in patients with extra-pancreatic NET (NCT02588170).

### REFERENCES

1. Cébe-Suarez S, Zehnder-Fjällman A, Ballmer-Hofer K. The role of VEGF receptors in angiogenesis; complex partnerships. Cellular and Molecular Life Sciences 2006; 63:601-15.
2. Daniele G, Corral J, Molife L R, de Bono J S. FGF Receptor Inhibitors: Role in Cancer Therapy. Current Oncology Reports 2012; 14:111-9.
3. Folkman J. Role of angiogenesis in tumor growth and metastasis. Semin Oncol 2002; 29:15-8.
4. Ellis L M, Hicklin D J. Pathways Mediating Resistance to Vascular Endothelial Growth Factor-Targeted Therapy. Clin Cancer Res 2008; 14:6371-5.
5. Sitohy B, Nagy J A, Dvorak H F. Anti-VEGF/VEGFR therapy for cancer: Reassessing the target. Cancer Research 2012; 72:1909-14.
6. Voron T, Colussi O, Marcheteau E, Pernot S, Nizard M, Pointet A L, et al. VEGF-A modulates expression of inhibitory checkpoints on CD8(+) T cells in tumors. The Journal of Experimental Medicine 2015; 212:139-48.
7. Katoh MASA. FGFR inhibitors: Effects on cancer cells, tumor microenvironment and whole-body homeostasis (Review). International Journal of Molecular Medicine 2016; 38:3-15.
8. Hutchison Medipharma Limited. Chemical structure and synthesis: U.S. Pat. No. 8,658,658 B2. 2014;
9. Motzer R J, Hutson T E, Tomczak P, Michaelson M D, Bukowski R M, Rixe O, et al. Sunitinib versus Interferon Alfa in Metastatic Renal-Cell Carcinoma. N Engl J Med 2007; 356:115-24.
10. Escudier B. Eisen T, Stadler W M, Szczylik C, Oudard Sp, Siebels M, et al. Sorafenib in Advanced Clear-Cell Renal-Cell Carcinoma. N Engl J Med 2007; 356:125-34.
11. Raymond E, Dahan L. Raoul J L, Bang Y J, Borbath I, Lombard-Bohas C, et al. Sunitinib Malate for the Treatment of Pancreatic Neuroendocrine Tumors. N Engl J Med 2011; 364:501-13.
12. Zhou A, Zhang W, Chang C, Chen X, Zhong D, Qin Q, et al. Phase I study of the safety, pharmacokinetics and antitumor activity of famitinib. Cancer Chemotherapy and Pharmacology 2013; 72:1043-53.
13. Oberg K, Knigge U, Kwekkeboom D, Perren A, on behalf of the ESMO Guidelines Working Group. Neuroendocrine gastro-entero-pancreatic tumors: ESMO Clinical Practice Guidelines for diagnosis, treatment and follow-up. Annals of Oncology 2012; 23:vii124-vii130.
14. Oberg K, Hellman P, Ferolla P, Papotti M, on behalf of the ESMO Guidelines Working Group. Neuroendocrine bronchial and thymic tumors: ESMO Clinical Practice Guidelines for diagnosis, treatment and follow-up. Annals of Oncology 2012; 23:vii120-vii123.
15. Wong M H, Chan D L, Lee A, Li B T, Lumba S, Clarke S J, et al. Systematic Review and Meta-Analysis on the Role of Chemotherapy in Advanced and Metastatic Neuroendocrine Tumor (NET). PLoS ONE 2016; 11:e0158140.
16. Yao J C, Hassan M, Phan A, Dagohoy C, Leary C, Mares J E, et al. One Hundred Years After Carcinoid: Epidemiology of and Prognostic Factors for Neuroendocrine Tumors in 35,825 Cases in the United States. Journal of Clinical Oncology 2008; 26:3063-72.
17. Yao J C, Shah M H, Ito T, Bohas C L, Wolin E M. Van Cutsem E, et al. Everolimus for Advanced Pancreatic Neuroendocrine Tumors. N Engl J Med 2011; 364:514-23.
18. Yao J C, Fazio N, Singh S, Buzzoni R, Carnaghi C, Wolin E, et al. Everolimus for the treatment of advanced, non-functional neuroendocrine tumours of the lung or gastrointestinal tract (RADIANT-4): a randomised, placebo-controlled, phase 3 study. The Lancet 2016; 387:968-77.
19. Gross S, Rahal R, Stransky N, Lengauer C, Hoeflich K P. Targeting cancer with kinase inhibitors. J Clin Invest 2015; 125:1780-9.

1. A method of treating a solid tumor in a patient comprising administration to the patient in need thereof a therapeutically effective amount of a compound that is an inhibitor of VEGFR 1, 2, and 3, FGFR 1, and CSF1R.

2. The method of claim 1, wherein the compound is N-(2-(dimethylamino)ethyl)-1-(3-((4-((2-methyl-1H-indol-5-yl)oxy)pyrimidin-2-yl)amino)phenyl)-methanesulfonamide.

3. The method of claim 1, wherein the solid tumor is a neuroendocrine tumor (NET).

4. A method of treating a solid tumor in a patient comprising administration to the patient in need thereof once daily N-(2-(dimethylamino) ethyl)-1-(3-((4-((2-methyl-1H-indol-5-yl)oxy)pyrimidin-2-yl)amino)phenyl)-methanesulfonamide in an amount ranging from 200 to 350 mg.

5. The method of claim 4, wherein the once daily N-(2-(dimethylamino)ethyl)-1-(3-((4-((2-methyl-1H-indol-5-yl)oxy)pyrimidin-2-yl)amino)phenyl)-methanesulfonamide is in an amount of 200, 300, or 350 mg.

6. The method of claim 4, wherein the solid tumor is NET.

7. The method of claim 4, wherein the method demonstrates ORR (objective response rate) of greater than 20.0% and DCR (disease control rate) of greater than 65.0%.

8. The method of claim 5, wherein the once daily N-(2-(dimethylamino)ethyl)-1-(3-((4-((2-methyl-1H-indol-5-yl)oxy)pyrimidin-2-yl)amino)phenyl)-methanesulfonamide is in an amount of 300 mg.

9. The method of claim 8, wherein the method demonstrates an ORR (objective response rate) of greater than 20.0%, DCR (disease control rate) of greater than 60.0%.

10. The method of claim 5, wherein the once daily N-(2-(dimethylamino)ethyl)-1-(3-((4-((2-methyl-1H-indol-5-yl)oxy)pyrimidin-2-yl)amino)phenyl)-methanesulfonamide is in an amount of 350 mg.

11. The method of claim 10, wherein the method demonstrates an ORR (objective response rate) of greater than 30.0%, DCR (disease control rate) of greater than 70.0%.

12. The method of claim 6, wherein the method demonstrates an ORR (objective response rate) of greater than 30.0%, DCR (disease control rate) of greater than 80.0%, and a median of PFS (progression-free survival) of greater than 15.0 months (95% CI: 10.3-NR).

13. A method of treating NET in a patient comprising administration to the patient in need thereof once daily N-(2-(dimethylamino) ethyl)-1-(3-((4-((2-methyl-1H-indol-5-yl)oxy)pyrimidin-2-yl)amino)phenyl)-methanesulfonamide in an amount of 300 mg.

14. The method of claim 2, wherein the solid tumor is a neuroendocrine tumor (NET).

15. The method of claim 5, wherein the solid tumor is NET.

16. The method of claim 5, wherein the method demonstrates ORR (objective response rate) of greater than 20.0% and DCR (disease control rate) of greater than 65.0%.

17. The method of claim 5, wherein the method demonstrates an ORR (objective response rate) of greater than 30.0%, DCR (disease control rate) of greater than 80.0%, and a median of PFS (progression-free survival) of greater than 15.0 months (95% CI: 10.3-NR).
